(19) United States
(12) Patent Application Publication
(10) Pub. No.: US 2011/0135773 A1
(43) Pub. Date: Jun. 9, 2011

(54) COMPOSITION FOR EXTERNAL SKIN APPLICATION CONTAINING PINE KNOT EXTRACT AND METHOD FOR PREPARATION OF THE SAME

(75) Inventors: Ho Sik Rho, Yongin-si (KR); Sujong Kim, Yongin-si (KR); Eun Joo Kim, Suwon-si (KR); Eunjeong Moon, Seoul (KR); Gayoung Cho, Yongin-si (KR); Sung II Park, Seoul (KR); Youn Jun Kim, Seoul (KR); Duck Hee Kim, Seoul (KR); Han Kon Kim, Suwon-si (KR)

(73) Assignee: Amorepacific Corporation, Seoul (KR)

(21) Appl. No.: 13/001,010

(22) PCT Filed: Jun. 23, 2009
(86) PCT No.: PCT/KR09/03369

§ 371 Date: Feb. 7, 2011

(30) Foreign Application Priority Data
Jun. 24, 2008 (KR) 10-2008-0059858

(51) Int. Cl. A61K 8/97; A61Q 19/08

(52) U.S. Cl. 424/770

(57) ABSTRACT

Disclosed is a composition for external skin application for preventing skin aging comprising pine knot extract as an effective ingredient. The composition prevents and improves wrinkles through of skin cell proliferation and collagen biosynthesis, and may be widely applied in the field of skin beauty care and cosmetics.

### TECHNICAL FIELD

This disclosure relates to a composition for external skin application containing pine knot extract as an effective ingredient. More particularly, it relates to a composition for external skin application for preventing skin aging which contains pine knot extract having superior skin cell proliferation and collagen biosynthesis facilitating effect as an effective ingredient.

### BACKGROUND ART

As the first defensive barrier of the human body, the skin provides protection for various organs from change of temperature and humidity and environmental stimulations e.g. UV or pollutants, as well as maintenance of homeostasis. However, excessive physical/chemical stimulations or stresses from outside or undernutrition result in deterioration of the skins normal functions and accelerate skin aging, e.g. loss of elasticity, cornification, wrinkle formation, etc.

In order to prevent skin aging and maintain healthy and elastic skin, cosmetic materials derived from natural substances have been used. For example, cosmetics containing physiologically active substances extracted from various plants or microorganisms have been used to maintain the skins intrinsic functions and prevent skin aging by activating skin cells.

However, most of existing cosmetic materials do not provide sufficient effects or induce adverse cutaneous reactions. Accordingly, there is a strong need for materials providing skin anti-aging effects without inducing adverse reactions.

According to Dongeui Bogam, pine knot has been used relieve rheumatism or arthritis from old times and, when brewed into a wine, it can strengthen weak legs. This suggests that pine knot may contain various active ingredients. Through consistent researches, the inventors have developed a composition for external skin application using pine knot.

### DISCLOSURE OF INVENTION

### Technical Problem

This disclosure is directed to providing a composition for external skin application for preventing skin aging which contains pine knot extract as an effective ingredient.

This disclosure is also directed to providing a composition for external skin application containing pine knot extract which is effective in facilitating skin cell proliferation and collagen biosynthesis.

This disclosure is also directed to providing a method for preparing a composition for external skin application containing pine knot extract.

### Technical Solution

The composition for external skin application according to this disclosure contains pine knot extract as an effective ingredient.

### Advantageous Effects

The composition containing pine knot extract according to this disclosure provides the effect of preventing skin aging through facilitation of skin cell proliferation and collagen biosynthesis and, thus, may be widely utilized in the field of skin beauty care and cosmetic

### MODE FOR THE INVENTION

Exemplary embodiments now will be described more fully hereinafter with reference to the accompanying drawings, in which exemplary embodiments are shown. This disclosure may, however, be embodied in many different forms and should not be construed as limited to the exemplary embodiments set forth therein. Rather, these exemplary embodiments are provided so that this disclosure will be thorough and complete, and will fully convey the scope of this disclosure to those skilled in the art. In the description, details of well-known features and techniques may be omitted to avoid unnecessarily obscuring the presented embodiments.

The terminology used herein is for the purpose of describing particular embodiments only and is not intended to be limiting of this disclosure. As used herein, the singular forms “a”, “an” and “the” are intended to include the plural forms as well, unless the context clearly indicates otherwise. Furthermore, the use of the terms a, an, etc. does not denote a limitation of quantity, but rather denotes the presence of at least one of the referenced item. The use of the terms “first”, “second” and the like does not imply any particular order, but they are included to identify individual elements. Moreover, the use of the terms first, second, etc. does not denote any order or importance, but rather the terms first, second, etc. are used to distinguish one element from another. It will be further understood that the terms “comprises” and/or “comprising”, or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof.

Unless otherwise defined, all terms (including technical and scientific terms) used herein have the same meaning as commonly understood by one of ordinary skill in the art. It will be further understood that terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

In an embodiment of this disclosure, a composition for external skin application contains pine knot extract as an effective ingredient. In an embodiment, the pine knot extract has the effect of facilitating skin cell (fibroblast) proliferation and facilitating collagen biosynthesis. In another embodiment, the composition for external skin application has the effect of preventing skin aging and preventing or improving wrinkles.

As used in this disclosure, the “pine knot” means the portion of a pine tree where there is a knot or branch, and includes the portion where the pine branch grows and the portion where the pine branch has been cut. At the portion where the pine branch grows, pine resin is collected. The portion where the pine branch has been cut turns pale yellow because of the pine resin.

In an embodiment, the pine knot extract may be contained in an amount of 0.0001 to 10 weight %, more particularly 0.001 to 5.0 weight %, based on the total weight of the composition. If the content of the pine knot extract is lower than the aforesaid range, the effect of facilitating skin cell proliferation and collagen biosynthesis may be slight. And, if the content exceeds the aforesaid range, the final formulation form may have undesirable color or odor.

This disclosure also provides a method for preparing the composition for external skin application. In an embodiment, a method for preparing the composition for external skin application comprises: (a) grinding pine knot; (b) extracting the ground pine knot by cold infusion or maceration using a low polar solvent; and (c) concentrating the extract of (b) under reduced pressure using a distillation apparatus equipped with a cooling condenser.

In an embodiment, the low polar solvent may be one or more solvent(s) selected from a group consisting of C₁-C₄ anhydrous or hydrated low alcohol (e.g. methanol or ethanol), acetone, ethyl acetate, diethyl acetate, diethyl ether, benzene, chloroform and hexane.

In another embodiment, a method for preparing the composition for external skin application may further comprise, after the extraction of pine knot using anhydrous or hydrated low alcohol or acetone, (b-1) performing re-extraction using a low polar solvent. The low polar solvent for the re-extraction of (b-1) may be one or more solvent(s) selected from a group consisting of ethyl acetate, diethyl ether, benzene, chloroform and hexane. Through the re-extraction, the content of the active ingredient may be maximized, impurities may be removed, stability of the extract may be improved, and the desired effect may be maximized.

Hereinafter will be given a more detailed description of the method for preparing the composition containing pine knot extract according to this disclosure. Pine knot is finely ground. The ground pine knot is subjected to dipping extraction by 5-20 equivalent volume of an extraction solvent based on the dry weight of the ground pine knot. After filtration, the filtrate is concentrated under reduced pressure. The extraction solvent may be a polar solvent such as water, low alcohol (e.g. methanol or ethanol), water-low alcohol mixture, acetone, 1,3-butylene glycol, n-propanol, isopropanol, n-butanol, etc., or a mixture thereof.

After dispersing the concentrate by adding water, the dispersion is agitated after adding the same volume of a low polar organic solvent such as ethyl acetate, chloroform, diethyl ether, etc. The organic layer is separated and an extract with superior activity is obtained by concentration under reduced pressure. The effective ingredient may be extracted by cold infusion or percolation for about 12-96 hours, or by immersing in C₁-C₄ anhydrous or hydrated low alcohol, ethyl acetate or diethyl ether at normal temperature of 4-25° C. for 3-20 days. Alternatively, maceration may be carried out at a temperature close to the reflux temperature of the solvent for about 5-24 hours, although the condition may be varied depending on the solvent and the maceration temperature.

In an embodiment, the composition may be used for preventing skin aging through facilitation of skin cell proliferation and collagen biosynthesis. The formulation form of the composition for external application is not particularly limited depending on the body portion to which it is applied. For example, the composition for external skin application may be a cosmetic composition applied on the skin in the form of softening lotion, nourishing lotion, massage cream, nourishing cream, pack or gel, or in a transdermal administration form such as lotion, ointment, gel, cream, patch or spray.

Further, the composition for external application in each formulation form may include ingredients other than the pine knot extract. The ingredients may be selected by those skilled in the art without difficulty considering the formulation form, purpose of use, or the like. A synergic effect may be obtained by the addition of the ingredients.

The examples and experiments will now be described. The following examples and experiments are for illustrative purposes only and not intended to limit the scope of this disclosure.

### EXAMPLE 1

Pine knot was washed with purified water, dried and finely ground into powder. The pine knot powder (100 g) was mixed with 50% ethanol aqueous solution (1 L). The mixture was extracted by boiling in an extractor equipped with a cooling condenser for 12 hours. The extract was filtered through 300 mesh filter cloth. The filtrate was kept at 4-15° C. for 7 days and filtered through Whatman No. 2 filter paper. The filtered extract was put in a 3 L separatory funnel and, after adding ethyl acetate (1 L), mixed well by shaking. The mixture was left at normal temperature. After complete separation into two layers, the upper layer (ethyl acetate layer) was collected. Extraction was performed two more times using a separatory funnel from the lower layer (aqueous layer). The collected upper layers were combined and concentrated at 50° C. under reduced pressure using a distillation apparatus equipped with a cooling condenser. After drying, 22.1 g (dry weight) of extract was obtained.

### COMPARATIVE EXAMPLE 1

Pine needle was washed with purified water, dried and finely ground into powder. The pine needle powder (100 g) was mixed with 50% ethanol aqueous solution (1 L). The mixture was extracted by boiling in an extractor equipped with a cooling condenser for 12 hours. The extract was filtered through 300 mesh filter cloth. The filtrate was kept at 4-15° C. for 7 days and filtered through Whatman No. 2 filter paper. The filtered extract was put in a 3 L separatory funnel and, after adding ethyl acetate (1 L), mixed well by shaking. The mixture was left at normal temperature. After complete separation into two layers, the upper layer (ethyl acetate layer) was collected. Extraction was performed two more times using a separatory funnel from the lower layer (aqueous layer). The collected upper layers were combined and concentrated at 50° C. under reduced pressure using a distillation apparatus equipped with a cooling condenser. After drying, 17.5 g (dry weight) of extract was obtained.

### COMPARATIVE EXAMPLE 2

Pine bark was washed with purified water, dried and finely ground into powder. The pine bark powder (100 g) was mixed with 50% ethanol aqueous solution (1 L). The mixture was extracted by boiling in an extractor equipped with a cooling condenser for 12 hours. The extract was filtered through 300 mesh filter cloth. The filtrate was kept at 4-15° C. for 7 days and filtered through Whatman No. 2 filter paper. The filtered extract was put in a 3 L separatory funnel and, after adding ethyl acetate (1 L), mixed well by shaking. The mixture was left at normal temperature. After complete separation into two layers, the upper layer (ethyl acetate layer) was collected. Extraction was performed two more times using a separatory funnel from the lower layer (aqueous layer). The collected upper layers were combined and concentrated at 50° C. under reduced pressure using a distillation apparatus equipped with a cooling condenser. After drying, 19.1 g (dry weight) of extract was obtained.

### COMPARATIVE EXAMPLE 3

Pine cone was washed with purified water, dried and finely ground into powder. The pine cone powder (100 g) was mixed with 50% ethanol aqueous solution (1 L). The mixture was extracted by boiling in an extractor equipped with a cooling condenser for 12 hours. The extract was filtered through 300 mesh filter cloth. The filtrate was kept at 4-15° C. for 7 days and filtered through Whatman No. 2 filter paper. The filtered extract was put in a 3 L separatory funnel and, after adding ethyl acetate (1 L), mixed well by shaking. The mixture was left at normal temperature. After complete separation into two layers, the upper layer (ethyl acetate layer) was collected. Extraction was performed two more times using a separatory funnel from the lower layer (aqueous layer). The collected upper layers were combined and concentrated at 50° C. under reduced pressure using a distillation apparatus equipped with a cooling condenser. After drying, 16.4 g (dry weight) of extract was obtained.

### TEST EXAMPLE 1

### Skin Cell Proliferation Effect of Pine Knot Extract

Skin cell proliferation effect of pine knot extract was tested. First, human normal fibroblasts were inoculated on a 96-well microplate, 1×10⁴ cells per each well. The inoculated cells were cultured in Dulbecco's modified Eagle's medium (DMEM) for 24 hours. The cells were further cultured for 24 hours after replacing the medium with serum-free DMEM containing 500 μg/mL of the pine knot extract of Example 1 or the extracts of Comparative Examples 1-3, respectively. After adding 5 mg/mL 3-(4,5-dimethylthiazol-2-yl)2,5-diphenyltetrazolium (MTT), 10 μL per each well, the medium was removed 4 hours later. After adding dimethyl sulfoxide, 100 μL per each well, absorbance was made at 570 nm using a microplate reader.

Cell proliferation effect was calculated by Equation 1, and the result is given in Table 1.

Cell proliferation effect(%)=[(Absorbance after treatment with extract−Absorbance of control group)/Absorbance of control group]×100 [Equation 1]

TABLE 1

| | Absorbance | Cell proliferation |
|---|---|---|
| Sample (500 μg/mL) | (570 nm) | effect (%) |
| Control group | 0.310 | — |
| Example 1 | 0.591 | 90.6% |
| Comparative Example 1 | 0.534 | 72.2% |
| Comparative Example 2 | 0.490 | 58.0% |
| Comparative Example 3 | 0.520 | 67.8% |

As seen in Table 1, the sample containing pine knot extract (Example 1) exhibited markedly superior cell proliferation effect for normal fibroblasts, as compared to the samples containing extracts from other portions of pine tree (Comparative Examples 1-3).

### TEST EXAMPLE 2

### Collagen Biosynthesis Facilitating Effect

Collagen biosynthesis facilitating effect of pine knot extract was tested as follows. Human normal fibroblasts were inoculated on a 96-well microplate, 1×10⁴ cells per each well. The inoculated cells were cultured in DMEM for 24 hours. The cells were then further cultured for 48 hours after replacing the medium with serum-free DMEM containing 500 μg/mL of the pine knot extract of Example 1 or the extracts of Comparative Examples 1-3, respectively. 24 hours before the final culturing, 50 μg/mL ascorbic acid was added to promote collagen synthesis. Then, after washing each well, followed by replacing the medium with serum-free DMEM again, the cells were further cultured for 24 hours. The supernatant of each well was collected and the amount of collagen synthesis was measured using a procollagen type I C-peptide (PICP) kit (Takara, Kyoto, Japan). The result is given in Table 2.

TABLE 2

| | Collagen synthesis |
|---|---|
| Sample (500 μg/mL) | amount (ng/2 × 10⁴) |
| Control group | 142 |
| Example 1 | 290 |
| Comparative Example 1 | 262 |
| Comparative Example 2 | 271 |
| Comparative Example 3 | 240 |

As seen in Table 2, the sample containing pine knot extract (Example 1) exhibited about 7-20% improved collagen biosynthesis facilitating effect, as compared to the samples containing extracts from other portions of pine tree (Comparative Examples 1-3).

The following formulation examples of the composition are provided for illustrative purposes only and not intended to limit the scope of this disclosure.

TABLE 3
[Formulation Example 1] Softening lotion (skin lotion)

| Ingredients | weight % |
|---|---|
| Pine knot extract | 0.5 |
| β-1,3-Glucan | 1.0 |
| Butylene glycol | 2.0 |
| Propylene glycol | 2.0 |
| Carboxyvinyl polymer | 0.1 |
| PEG-12 nonyl phenyl ether | 0.2 |
| Polysorbate 80 | 0.4 |
| Ethanol | 10.0 |
| Triethanolamine | 0.1 |
| Antiseptic, pigment and perfume | adequate |
| Purified water | to 100 |

TABLE 4
[Formulation Example 2] Nourishing lotion (milk lotion)

| Ingredients | weight % |
|---|---|
| Pine knot extract | 0.5 |
| β-1,3-Glucan | 1.0 |
| Beeswax | 4.0 |
| Polysorbate 60 | 1.5 |
| Sorbitan sesquioleate | 1.5 |
| Liquid paraffin | 0.5 |
| Caprylic/capric triglyceride | 5.0 |
| Glycerine | 3.0 |
| Butylene glycol | 3.0 |
| Propylene glycol | 3.0 |
| Carboxyvinyl polymer | 0.1 |
| Triethanolamine | 0.2 |
| Antiseptic, pigment and perfume | adequate |
| Purified water | to 100 |

TABLE 5
[Formulation Example 3] Nourishing cream

| Ingredients | weight % |
|---|---|
| Pine knot extract | 1.0 |
| β-1,3-Glucan | 5.0 |
| Beeswax | 10.0 |
| Polysorbate 60 | 1.5 |
| PEG 60 hydrogenated castor oil | 2.0 |
| Sorbitan sesquioleate | 0.5 |
| Liquid paraffin | 10.0 |
| Squalane | 5.0 |
| Caprylic/capric triglyceride | 5.0 |
| Glycerine | 5.0 |
| Butylene glycol | 3.0 |
| Propylene glycol | 3.0 |
| Triethanolamine | 0.2 |
| Antiseptic, pigment and perfume | adequate |
| Purified water | to 100 |

TABLE 6
[Formulation Example 4] Massage cream

| Ingredients | weight % |
|---|---|
| Pine knot extract | 1.0 |
| β-1,3-Glucan | 3.0 |
| Beeswax | 10.0 |
| Polysorbate 60 | 1.5 |
| PEG 60 hydrogenated castor oil | 2.0 |
| Sorbitan sesquioleate | 0.8 |
| Liquid paraffin | 40.0 |
| Squalane | 5.0 |
| Caprylic/capric triglyceride | 4.0 |
| Glycerine | 5.0 |
| Butylene glycol | 3.0 |
| Propylene glycol | 3.0 |
| Triethanolamine | 0.2 |
| Antiseptic, pigment and perfume | adequate |
| Purified water | to 100 |

TABLE 7
[Formulation Example 5] Pack

| Ingredients | weight % |
|---|---|
| Pine knot extract | 0.5 |
| β-1,3-Glucan | 1.0 |
| Polyvinyl alcohol | 13.0 |
| Sodium carboxymethyl cellulose | 0.2 |
| Glycerine | 5.0 |
| Allantoin | 0.1 |
| Ethanol | 6.0 |
| PEG-12 nonyl phenyl ether | 0.3 |
| Polysorbate 60 | 0.3 |
| Antiseptic, pigment and perfume | adequate |
| Purified water | to 100 |

TABLE 8
[Formulation Example 6] Gel

| Ingredients | weight % |
|---|---|
| Pine knot extract | 0.05 |
| β-1,3-Glucan | 0.1 |
| Ethylenediamine sodium acetate | 0.05 |
| Glycerine | 5.0 |
| Carboxyvinyl polymer | 0.3 |
| Ethanol | 5.0 |
| PEG-60 hydrogenated castor oil | 0.5 |
| Triethanolamine | 0.3 |
| Antiseptic, pigment and perfume | adequate |
| Purified water | to 100 |

TABLE 9
[Formulation Example 7] Ointment

| Ingredients | weight % |
|---|---|
| Pine knot extract | 2.0 |
| β-1,3-Glucan | 10.0 |
| Beeswax | 10.0 |
| Polysorbate 60 | 5.0 |
| PEG 60 hydrogenated castor oil | 2.0 |
| Sorbitan sesquioleate | 0.5 |
| Vaseline | 5.0 |
| Liquid paraffin | 10.0 |
| Squalane | 5.0 |
| Shea butter | 3.0 |
| Caprylic/capric triglyceride | 5.0 |
| Glycerine | 10.0 |
| Propylene glycol | 10.2 |
| Triethanolamine | 0.2 |
| Antiseptic, pigment and perfume | adequate |
| Purified water | to 100 |

TABLE 10
[Formulation Example 8] Topical application form (gel ointment)

| Ingredients | weight % |
|---|---|
| Pine knot extract | 3.0 |
| β-1,3-Glucan | 10.0 |
| Polyacrylic acid (Carbopol 940) | 1.5 |
| Isopropanol | 5.0 |
| Hexylene glycol | 25.0 |
| Triethanolamine | 1.7 |
| Deionized water | to 100 |

TABLE 11
[Formulation Example 9] Topical application form (patch)

| Ingredients | weight % |
|---|---|
| Pine knot extract | 1.0 |
| β-1,3-Glucan | 3.0 |
| Hexylene glycol | 20.0 |
| Diethylamine | 0.7 |
| Polyacrylic acid (Carbopol 934P) | 1.0 |
| Sodium sulfite | 0.1 |
| Polyoxyethylene lauryl ether (E.O = 9) | 1.0 |
| Polyhydroxyethylene cetyl stearyl ether | 1.0 |
| (Cetomacrogol 1000) | |
| Viscous paraffin oil | 2.5 |
| Caprylic acid ester/capric acid ester (Cetiol | 2.5 |
| LC) | |
| Polyethylene glycol 400 | 3.0 |
| Deionized water | To 100 |

While the exemplary embodiments have been shown and described, it will be understood by those skilled in the art that various changes in form and details may be made thereto without departing from the spirit and scope of this disclosure as defined by the appended claims.

In addition, many modifications can be made to adapt a particular situation or material to the teachings of this disclosure without departing from the essential scope thereof. Therefore, it is intended that this disclosure not be limited to the particular exemplary embodiments disclosed as the best mode contemplated for carrying out this disclosure, but that this disclosure will include all embodiments falling within the scope of the appended claims.

1. A composition for external skin application containing pine knot extract as an effective ingredient.

2. The composition for external skin application according to claim 1, wherein the pine knot extract is included in an amount of 0.0001 weight % to 10 weight % based on the total weight of the composition.

3. The composition for external skin application according to claim 1, wherein the pine knot extract is effective for skin cell proliferation.

4. The composition for external skin application according to claim 1, wherein the pine knot extract is effective for facilitating collagen biosynthesis.

5. The composition for external skin application according to claim 1, which is for preventing skin aging.

6. The composition for external skin application according to claim 1, which is for preventing or improving wrinkles.

7. A method for preparing a composition for external skin application containing pine knot extract as effective ingredient, comprising:
grinding pine knot;
extracting the ground pine knot by cold infusion or maceration using a low polar solvent; and
concentrating the extract under reduced pressure using a distillation apparatus equipped with a cooling condenser.

8. The method for preparing a composition for external skin application according to claim 7, wherein the low polar solvent is one or more solvent(s) selected from a group consisting of C₁-C₄ anhydrous or hydrated low alcohol, acetone, ethyl acetate, diethyl acetate, diethyl ether, benzene, chloroform and hexane.

9. The method for preparing a composition for external skin application according to claim 7, which further comprises, after said extracting:
performing re-extraction of the extract using one or more solvent(s) selected from a group consisting of ethyl acetate, diethyl ether, benzene, chloroform and hexane.

10. A method for preventing skin aging comprising administering an effective amount of the composition according to claim 1 to a subject in such need.

11. The method according to claim 10, which prevents skin aging by facilitating skin cell proliferation and collagen biosynthesis.

12. The method according to claim 10, wherein the composition according to claim 1 is administered transdermally.

13. A method for preventing or improving wrinkles comprising administering an effective amount of the composition according to claim 1 to a subject in such need.
